(19) United States
(12) Patent Application Publication
(10) Pub. No.: US 2006/0252049 A1
(43) Pub. Date: Nov. 9, 2006

(54) Growth-promoting and immunizing subcutaneous implant

(76) Inventors: Richard O. Shuler, Alpharetta, GA (US); Ronald Cravens, Leawood, KS (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/121,535

(22) Filed: May 4, 2005

(51) Int. Cl. C12Q 1/68; G01N 33/53

(52) U.S. Cl. 435/6; 435/7.1

(57) ABSTRACT

The present invention relates generally to an implant for use in animals. More particularly, the present invention relates to an implant including a pharmaceutical agent and a biological agent for use in animals to promote growth, regulate metabolism and prevent illness or disease. A method for promoting growth and immunity in animal using an implant including a pharmaceutical agent and a biological agent. The implant is preferably administered to an animal subcutaneously.

### BACKGROUND OF THE INVENTION

The present invention relates generally to an implant for use in animals. More particularly, the present invention relates to an implant including a pharmaceutical agent and a biological agent for use in animals to promote growth, regulate metabolism and prevent illness or disease. The present invention is particularly suitable for use in livestock such as beef cattle, dairy cattle, swine, poultry, sheep, and the like.

Subcutaneous implantation of pharmaceuticals and medical devices is a well-accepted procedure and has been widely adopted for therapeutic, health and growth enhancement purposes for livestock and companion animals, humans, and even certain wild animals, such as those maintained in nature preserves, parks and zoos. In the case of animals that are raised for slaughtering and human consumption, the increasing demand for edible protein has resulted in a particular interest in promoting physiological growth and weight gain in these animals. Disease prevention also plays an important role in the raising of animals intended for human or domestic animal consumption to ensure a safe source of protein.

Pharmaceuticals are commonly administered as solid compressed pellets that are injected by an implanter equipped with a hypodermic needle. The needle is used to make a surface self-sealing and non-coring implant-receiving puncture beneath the skin of the ear of an animal. Small pellets of pharmaceutical are forced through the needle and left under the skin as the needle is removed from the ear. The ears are commonly discarded in slaughtering, such that no unabsorbed residues of such pellets will end up in food products intended for humans or domestic animals. One skilled in the art will appreciate that, although such implants are normally made in the ear, other areas of the animal that are not used for consumption and are discarded may be used for an implant site. Similar therapeutic procedures may be employed to implant drug delivery devices such as controlled release osmotic pumps in humans and animals as well as transponder devices in animals.

In the case of farm animals, the pellets are normally implanted while an animal is confined in a chute. An ear is grasped in one hand, and an implanter device having a large hypodermic needle is used to puncture the hide and subcutaneously inject a pellet dose into an implant receiving puncture. The implanting must be done carefully to insure that the pellets are properly placed and that no pellet remains extending from the puncture outside the hide. The procedure must be carried out quickly since the animals are not entirely cooperative and may shake their heads to free the held ear.

U.S. Pat. No. 5,522,797 (hereinafter “the '797 patent”), and entitled Slide Action Veterinary Implanter, which patent is hereby incorporated by reference to the extent permitted by law, discloses an implanter which employs a slide action mechanism to retract an impeller, store an impeller driving force in a spring in cooperation with a latch mechanism, reset a trigger, and advance a pellet magazine, all by a single trigger actuated reciprocation of the slide mechanism. Operation of the trigger also forces the pellets from the magazine through the needle and under the skin of the animal.

### BRIEF SUMMARY OF THE INVENTION

Accordingly, the present invention is directed to a growth-promoting and immunizing implant for use in animals. The preferred implant includes an effective amount of a biological agent and an effective amount of a pharmaceutical agent. The present invention is also directed to a method for promoting growth and immunity in animals wherein the method includes the steps of providing an implant including an effective amount of a biological immunizing agent, and an effective amount of a pharmaceutical agent, and then administering the implant to an animal.

### DETAILED DESCRIPTION OF THE INVENTION

While the present invention may be embodied in many different forms, there is discussed herein a specific embodiment with the understanding that the present disclosure is to be considered only as an exemplification of the principles of the invention and is not intended to limit the invention to the embodiments illustrated.

As used herein, the terms “agent” or “drug” may be used interchangeably and broadly to include any compound, composition of matter or mixture thereof that can be administered to an animal to produce a useful result.

As used herein, the term “effective amount” as applied to an agent or drug refers to that amount which is sufficient to effect the desired change in the subject animal. For example, where the desired effect is increased weight gain in livestock, the “effective amount” is a “livestock weight gain-promoting” amount and will vary depending on the agent used as well as the species of animal subject. In another example, where the desired effect is stimulation of a protective immune response to a biological agent, the “effective amount” of an agent is calibrated to the biologically relevant response from the animal which could include, but is not limited to, antibody, cell mediated, specific or non-specific immunity. Moreover, the “effective amount” of an agent also includes those amounts defined by USDA/APHIS/CVB and/or detailed in 9-CFR.

As used herein, the term “implant” may be used to refer to a single pellet for suitable implantation or a plurality of such pellets wherein the pellet or pellets include an effective amount of at least one agent or drug.

As used herein, the term “animal” may be a farm animal, domestic animal, wild animal, or a human.

In general, the present invention is directed to an implant including an effective amount of a pharmaceutical agent and an effective amount of a biological agent for use in animals to promote growth and prevent illness or disease. The preferred implant is designed for subcutaneous implantation but may alternatively be administered to other body cavities, for example, vaginally, nasally, sublingually, and the like. In a preferred administration method, the implant of the present invention is placed subcutaneously in the animal subject's ear. Alternative sites of subcutaneous administration include the nape of the subject's neck and the axillary region. While a particular implanting apparatus is discussed above by way of example and explanation, one skilled in the art will appreciate that any implanting device now known or hereafter developed that would be suitable for implantation of the implants of the present invention may be used.

In a preferred embodiment, the biological agent is present in an amount of from about 1-100% by weight per pellet or implant, more preferably from about 50-99% by weight, and most preferably from about 60-75% by weight. However, any range or limits within the ranges specifically set forth, or as defined by USDA/APHIS/CVB, may be used to produce an effective amount of biological agent depending upon the type of biological agent being administered, the type of species receiving such administration and various other factors.

A biological agent is preferably a USDA-licensed material that stimulates an immune response in the animal. Specifically, when administered to an animal, the biological agent of the present invention will cause the formation of antibodies or induce other resistance mechanisms by the animal. Live or killed viruses, live or killed bacteria, live or killed protozoa, and detoxified toxins are all well known biological agents and particularly useful ingredients in vaccines, bacterins, toxoids, bacterin-toxoids, and mixtures thereof, that are used to protect animals against specific diseases.

Vaccines can contain either a killed or living virus. A killed vaccine may include wild (pathogenic) or attenuated viruses while living vaccines usually include only attenuated viruses. Vaccines can also include living bacteria whereas bacterins can include killed bacteria. Toxoids are biologically active materials included alone as immunogens. Bacterin-toxoids are a suspension of killed bacteria along with toxoids.

The preferred biological agents for use in the present invention include, by way of example but not limitation, the following: infectious bovine rhinotracheitis virus, bovine viral diarrhea virus, bovine parainfluenza 3 virus, bovine respiratory syncytial virus, Haemophilus somnus, Mannheimia haemolytica, Pasteurella multocida, Leptospira spp., Campylobacter fetus, Clostridium spp., rotavirus, coronavirus, Escherichia coli, Moraxella bovis, Bordetella bronchiseptica, Erysipelothrix rhusiopathiae, Actinobacillus pleuropneumoniae, Mycoplasma hyopneumoniae, Mycoplasma bovis, Mycoplasma dispar, porcine respiratory reproductive syndrome virus, porcine parvovirus, transmissible gastroenteritis virus, pseudorabies virus, Salmonella spp., Lawsonia ssp., Coccidia spp., Anaplasma spp., Babbesia spp., canine parvovirus, canine adenovirus, canine distemper, canine parainfluenza, rabies, feline leukemia, feline vital rhinotracheitis, feline calivirus, feline panleukopenia, Chlamydia psittaci, combinations and mixtures thereof, and other biological agents currently known and hereafter identified immunogenic and protective proteins, known and hereafter identified nucleic acid segments or subsegments, known and hereafter identified soluble and insoluble extracts of cells, cell cultures, viruses, bacteria and bacterial culture material and mixtures thereof.

The preferred pharmaceutical agent for use in the present invention is a compound useful for effecting some beneficial change in the animal to which the agent is administered. Preferred pharmaceutical agents are present in the implant of the present invention in an amount of from about 1-100% by weight per pellet or implant, more preferably from about 50-99% by weight, and most preferably from about 60-75% by weight. However, any range or limits within the ranges specifically set forth, or as defined by the FDA, may be used to produce an effective amount of pharmaceutical agent depending upon the type of pharmaceutical agent being administered, the type of species receiving such administration and various other factors. Preferred pharmaceutical agents include, but are not limited to, parasiticides, pesticides, germicides, biocides, fungicides, insecticides, antioxidants, growth promotants, growth inhibitors, disinfectants, sterilization agents, catalysts, nutrients, vitamins, steroid hormones, prostaglandins, antibiotics, anti-inflammatory agents, chemotherapeutic agents, cardiovascular agents, antihypertensive agents, estrus suppressors, fertility promoters, somatotropins, and gonadotropins.

Particularly preferred parasiticides may include polyketide ivermectins, such as ivermectin, doramectin, moxidectin, eprinomectrin, and abamectin, the milbemycins and milbemycin oximes, fenbendazole, oxfendazole and luferon. As used herein, the term “parasiticide” is intended to include parasiticides as noted above and other compositions that operably function as parasiticides in combating infestation and preventing reinfestation by internal and external parasites.

Particularly preferred growth promotants may include progesterone, estradiol and derivatives thereof including estradiol benzoate, trenbolone acetate, and zeranol. As used herein, the term “growth promotant” is intended to include such agents as noted above and other compositions that operably function under the present invention to promote physiological growth.

A wide range of active ingredients may be employed as the antibiotic agent, such as macrolide antibiotics, especially tylosin and its salts, penicillin and derivatives thereof, tetracycline and its derivatives including oxytetracycline and their salts. Particularly preferred antibiotics include tylosin tartrate, tylosin, oxytetracycline, neomycin, tilmicosin phosphate, ceftiofur hydrochloride, ceftiofur sodium, and sulfadimethoxine. As used herein, the term antibiotic is intended to include the antibiotics noted above and other compositions that operably function like antibiotics in preventing infection and inflammation. Such antibiotics can also include bacteriostats, such as alcohols and glycols, anti-inflammatory agents, and other suitable antibacterial, bacteriostat, anti-inflammatory or combination thereof.

Particularly preferred steroid hormones include levonorgestrel, estradiol 17β, testosterone, testosterone propionate, ethynyl estradiol, and the like. Particularly preferred estrus suppressing compositions may include melengesterol acetate, norgestomet and other progestins.

In addition to those described above, pharmaceutical agents may include inorganic and organic macromolecular bioactive agents of bioactive origin. Organic and inorganic active agents may include but are not limited to acetylcholine esterase inhibitors, aminoglycocides, angiotensin converting enzyme inhibitors, antiarrhythmics, antibacterial agents, anticancer agents, antidepressants, antidiabetics, antiepileptics, anti-viral agents, antihistamines, antihypertensives, antinauseants, antiprostaglandins, antirheumatics, antiseptics, barbiturates, beta-blockers, betalactamase inhibitors, calcium channel blockers, cardiac glycosides, cephalosporins, immune reagents, immunostimulators, immuno-suppressives, liposaccharide complexing agents, methylxanthines, minerals, O-beta-hydroxyethylated rutins, propxyphenes, salicyclates, tetracyclin compounds, vasodilators, acetaminiophen, acetazolamide, acetophenetidin, achromycine hydrochloride, bendofluazide, benzthiozide, betamethasone, calcium and salts, thereof including, leucovorin calcium, carbamazepine, clindamycin, chlorpropamide, chlorothalidone, chlorothiazide, clofibrate, cortisone acetate, cyclopenthiazide, dexamethazone, dextroamphetamine sulphate, diclofenac sodium, dioxin, dimethindene and salts thereof, diprophylline, disopyramide and salts thereof, dipyrone, doxycycline, fenbufen, fenoprofen, ferrous fumarate, flurbiprofen, frusemide, furosemide, glibenclamide, haloperidol, hydralazine, hydrochloride hydrochlorothiazide, hydroflumethiazide ibuprofen, indomethacin, indoprofen, iron salts, kanamycin, ketoprofen, L-Dopa, lithium salts, metaclopramide, methazolamide, methotrexate, fluoro-uracil, methotrexate sodium, methyl Dopa, metronidazole, minocyclin hydrochloride, mofebutazone, morphine, naproxen, nifedipine, oxyphenbutazone, penicillin, peridinol and salts thereof, phenylbutazone, phenobarbital, phenylpropanolamine, phenytoin, pindolol, piroxicam, pirprofen, potassium chloride, prazosin, propanolol, proxyphilline, pyrvinium emboate, quinidine, reserpine, salicylamide, salicyl salicyclic acid, sodium fluoride, spironolactone, sulfadiazine, sulfamerazine, tetracyclin compounds, tolbutamide, trihexylphenidyl hydrochloride, triethoprim, valproic acid, vancomycin, zoxazolamine, carbonic anhydrase inhibitors, anti-glaucoma agents, benzalkonium chloride, benzocaine, amilorid, those materials that act upon the central nervous system such as hypnotics, sedatives, psychic energizers, tranquillizers, anticonvulsants, muscle relaxants, anti-Parkinson agents, analgesics, steroidal anti-inflammatories, anti autoimmune agents, local and systemic anesthetics, hormonal agents such as contraceptives, sympathomimetrics, parasympathomimetrics, lipid regulating agents, anti-androgenics, antiparasitics, neoplastics, anti-AIDS agents, mutagens, teratogens, hypoglycemic, nutritionals, fats, ophthalmics, otolaryngolmics, electrolytes, diagnostic agents, diuretics, nonsteroidal anti-inflammatories such as aspirin, ibuprofen, antihistamines such as diphenhydramine, chlomethazine, clemastine, hydroxyzine, terfenadine promethazine, astemizole, loratadine, mast cell stabilizers such as cromolyn sodium, bronchodilators such as metaproterol sulphate, isoetharine hydrochloride, theophylline, albuterol, epinedrine, norepinedrine, adrenaline, noradrenaline, corticosteroids such as prednisone, prednisolone, hydrocortisone, cortisone acetate, flunisolide and triamincinolone acetate, anti cholesterol agents, estradiol, progesterones, testosterone, amino acids, thyroxine, peptides such as enkephalins, histamine, fatty acids and fatty acid derivatives such as prostaglandins E2 etc., inositol phosphates, gamma-aminobutyric acid, ketone bodies, acetylcholine, and mixtures thereof.

Macromolecular bioactive agents also include but are not limited to protein, DNA, carbohydrates and mixtures thereof. This may include immunoglobulins G, M, A, D and E and their fragments and sub-chains, hormones such as insulin, somatotropins, growth hormones, somatomedins, erythromycin, adrenocorticotropic hormone (ACTH), parahormone, Follicle stimulating hormone, inhibin, renin, Leuteinizing hormone, Thyroid stimulating hormone, hypothalamic releasing hormones such as LH releasing factor, TSH releasing factor, gastrointestinal hormones such as gastrin, cholecystokinin, etc., vasopressin (ADH), somatostatin, immunomodulators, immunostimulators and immunoinhibitors such as cytokines including Interferons alpha beta, gamma, etc. and Interleukins 1, 2, 3, 4, etc., tumor necrosis factor alpha, beta, etc., colony stimulating factors (CSF) and growth factors such as Granulocyte CSF Macrophage CSF, Granulocyte-Macrophage CSF, Epidermal Growth Factor, Fibroblast Growth Factor, Nerve Growth Factors, cell chemotactic factors, antihemophilic factors such as Factor VIII, surface receptors and co-receptors, mineral oils, detergents, surfactants including but not limited to nonionic block polymer surfactants such as polyoxypropylene, polyoxyethylene, pluronic, saponin, immunomodulators, immunostimulators and immunoinhibitors, allergen source material, allergen extracts, denatured immunoglobulin receptors, and mixtures thereof.

The preferred implant of the present invention is biodegradable in the target animal and presents a controlled release of the active ingredients. The desired controlled release may be either immediate release of the active ingredients or a sustained release over a long period of time. One skilled in the art will appreciate that any known or hereafter developed methods for controlling such release, whether immediate or sustained, are suitable for use in the present invention.

It will also be appreciated by one skilled in the art that any suitable excipients may be used in the composition of the implants of the present invention. Preferred amounts of excipients include from about 0.1% to 50% by weight, most preferably about 1.0% to 40% by weight, and most preferably from about 1% to 25% by weight. However, any range or limits within the ranges specifically set forth may be used to produce an effective amount of excipient depending upon the type of biological agent or pharmaceutical agent being administered, the type of species receiving such administration and various other factors. Preferred excipients include, but are not limited to, starch, talc, glucose, sucrose, gelatin, malt, rice, flour, chalk, silica gel, calcium stearate, sodium stearate fumarate, stearic acid, sodium lauryl sulfonate, sodium stearate, glycerol monostearate, sodium chloride, polyethylene glycol, polyoxyethylene, glycerol behenate, hydrogenated vegetable oils, magnesium stearate, precipitated or fumed silicas, soidum starch glycolates, calcium phosphate, caldium carbonate, dextrins, polyvinyl pyrrolidone, polylactic acid, magnesium aluminum silicates, cellulose and its derivatives, especially ethylcellulose and microcrystalline cellulose, sodium carboxymethylcellulose, lactose, dried skim milk, derivatives thereof, and mixtures thereof. Preferred implants include excipients such as polyethylene glycol and tablet lubricants such as magnesium stearate and croscarmellose sodium.

The implants may also include a wide range of additives including, but not limited to, inert and functional fillers, glidants, disintegrants, lubricants, adjuvants, antibiotic preservatives, polymeric supports, binders, coloring agents, and mixtures thereof, to facilitate application, to control release, to stabilize the composition, to color code individual pellets, and for other reasons well known in the art. Preferred amounts of additives, singly or in combination, include from about 0.1% to 50% by weight, most preferably about 1.0% to 40% by weight, and most preferably from about 1% to 25% by weight. Preferred inert fillers include lactose, mannitol, dextrate, dextrose, fructose, sucrose, galactose, maltose, sorbitol, dextran, dextrin, calcium carbonate, calcium sulfate, dicalcium phosphate, and mixtures thereof. Functional fillers may include alginic acid, cellulosics such as hydroxypropyl cellulose (HPC), hydroxypropyl methylcellulose (HPMC), oxidized cellulose (OC), microcrystalline cellulose (MCC), ethyl cellulose (EC), hydroxyethyl cellulose (HEC), methyl cellulose (MC), carboxymethyl cellulose (CMC), cellulose acetate (CA), cellulose acetate butyrate (CAB), cellulose acetate propionate (CAP), cellulose sodium phosphate (CSP), cellulose triacetate (CTA), cellulose acetate phthalate (C-A-P), hydroxypropyl methylcellulose phthalate (HPMCP), cellulose acetate trimellitate (C-A-T), hydroxypropyl methylcellulose acetate succinate (HPMCAS), sodium carboxymethyl cellulose, polyanhydrides, polymethyl merthacrylate, polylactides, polyglycolides, carbomer, gellan gum, sodium alginate, acrylic copolymers, glyceryl monostearate, zein, cholesterol, agarose, chitosan, xanthan gum, polyethylene glycol (PEG), gelatin, povidone, natural gum, and mixtures thereof. Suitable glidants may include precipitated silica, fumed silica. Suitable disintegrants include sodium starch glycolate, crospovidone, croscarmellose sodium. Suitable lubricants include stearic acid, magnesium stearate, calcium stearate, sodium stearyl fumarate, glyceryl monostearate, triglyceride esters. Suitable adjuvants include aluminum hydroxide, saponin, dimethyl dioctadecyl ammonium bromide (DDA bromide), and bacterial extracts.

In accordance with the present invention, it will be appreciated that one or more biological agents and one or more pharmaceutical agents could be mixed together and incorporated into a single pellet; however, because each of the agents may be absorbed at different rates or require different carriers, there may also be a different pellet for each of the agents used. It is also within the scope of the present invention to have a single elongate or multiple shorter pellets containing mixtures of two or more agents or to have some agents in separate pellets injected with other agents that are mixed and formed into a common pellet.

The present invention is further illustrated by the following examples, which are not to be construed in any way as imposing limitations upon the scope thereof On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Two types of pellets, including biological agent pellets, are formulated to have different characteristics with respect to release of active ingredients. The first type is an immediate release and the second type is a controlled or sustained release, depending on the nature of the infection to be controlled. The following formulation provides relatively immediate release of active ingredient to the site of the implant receiving puncture:

### Formula I

90% by weight infectious bovine rhinotracheitis

8.0% by weight polyethylene glycol

1.5% by weight magnesium stearate

0.5% by weight croscarmellose sodium

The following formulation provides release of active ingredients to the site of the implant receiving puncture over a period of two to five days:

### Formula II

90% by weight oxytetracycline

8.0% by weight polyethylene glycol

2.0% by weight magnesium stearate

### EXAMPLE 2

Pellets containing the biological agent bovine viral diarrhea virus are produced according the following formulation:

26 mg bovine viral diarrhea virus

12.5 mg polyethylene glycol

0.5 mg magnesium stearate

The pellets are produced by compression on a rotary tablet press and twenty-one cattle are implanted with pellets including progesterone and estradiol benzoate pharmaceutical agents. Sixteen of the larger group of twenty-one cattle are implanted with one pellet of the biological agent of Formula III. The remaining five cattle receive the pharmaceutical implant pellets only and were not implanted with the biological agent pellet. The five cattle that did not receive the biological agent pellet served as the control group. A dose of Actinomyces pyogenese is administered to the exterior of the implant site of each of the twenty-one cattle in order to try to initiate infection in the implant-receiving puncture. After ten days, the implant sites are checked for abscess formation. The control cattle exhibit an 80% rate of abscess formation whereas the cattle implanted with the biological agent pellet of Formula III in addition to the pharmaceutical agent pellet exhibit only a 33% incidence of abscess formation.

What is claimed is:

1. A growth-promoting and immunizing implant for use in animals comprising:
an effective amount of at least one biological agent; and
an effective amount of at least one pharmaceutical agent.

2. The implant of claim 1, said effective amount of said biological agent being from about 1-100% by weight of said implant.

3. The implant of claim 1, said effective amount of said biological agent being from about 50-99% by weight of said implant.

4. The implant of claim 1, said effective amount of said biological agent being from about 60-75% by weight of said implant.

5. The implant of claim 1, wherein said biological agent is a compound that stimulates an immune response in an animal.

6. The implant of claim 5, wherein said biological agent is selected from the group consisting of vaccines, bacterins, toxoids, bacterin-toxoids, and mixtures thereof.

7. The implant of claim 6, wherein said biological agent is selected from the group consisting of infectious bovine rhinotracheitis virus, bovine viral diarrhea virus, bovine parainfluenza 3 virus, bovine respiratory syncytial virus, Haemophilus somnus, Mannheimia haemolytica, Pasteurella multocida, Leptospira spp., Campylobacterfetus, Clostridium spp., rotavirus, coronavirus, Escherichia coli, Moraxella bovis, Bordetella bronchiseptica, Erysipelothrix rhusiopathiae, Actinobacillus pleuropneumoniae, Mycoplasma hyopneumoniae, Mycoplasma bovis, Mycoplasma dispar, porcine respiratory reproductive syndrome virus, porcine parvovirus, transmissible gastroenteritis virus, pseudorabies virus, Salmonella spp., Lawsonia ssp., Coccidia spp., Anaplasma spp., Babbesia spp., canine parvovirus, canine adenovirus, canine distemper, canine parainfluenza, rabies, feline leukemia, feline vital rhinotracheitis, feline calivirus, feline panleukopenia, Chlamydia psittaci, combinations and mixtures thereof.

8. The implant of claim 1, said effective amount of said pharmaceutical agent being from about 1-100% by weight of said implant.

9. The implant of claim 1, said effective amount of said pharmaceutical agent being from about 50-99% by weight of said implant.

10. The implant of claim 1, said effective amount of said pharmaceutical agent being from about 60-75% by weight of said implant.

11. The implant of claim 1, wherein said pharmaceutical agent is selected from the group consisting of parasiticides, pesticides, germicides, biocides, fungicides, insecticides, antioxidants, growth promotants, growth inhibitors, disinfectants, sterilization agents, catalysts, nutrients, vitamins, steroid hormones, prostaglandins, antibiotics, anti-inflammatory agents, chemotherapeutic agents, cardiovascular agents, antihypertensive agents, estrus suppressors, fertility promotors, somatotropins, gonadotropins, and mixtures thereof.

12. The implant of claim 11, wherein said parasiticide is selected from the group consisting of polyketide ivermectins, milbemycins, milbemycin oximes, fenbendazole, oxfendazole, luferon, and mixtures thereof.

13. The implant of claim 11, wherein said growth promotant is selected from the group consisting of progesterone, estradiol, estradiol benzoate, trenbolone acetate, zeranol, derivatives thereof, and mixtures thereof.

14. The implant of claim 11, wherein said antibiotic is selected from the group consisting of macrolide antibiotics, penicillin, tetracycline, derivatives thereof, and mixtures thereof.

15. The implant of claim 14, wherein said antibiotic is selected from the group consisting of tylosin, tylosin tartrate, oxytetracycline, neomycin, tilmicosin phosphate, ceftiofur hydrochloride, ceftiofur sodium, sulfadimethoxine, derivatives thereof, and mixtures thereof.

16. The implant of claim 11, wherein said antibiotic is selected from the group consisting of bacteriostats, anti-inflammatory agents, and mixtures thereof.

17. The implant of claim 11, wherein said steroid hormone is selected from the group consisting of levonorgestrel, estradiol 17β, testosterone, testosterone propionate, ethynyl estradiol, derivatives thereof, and mixtures thereof.

18. The implant of claim 11, wherein said estrus suppressor is selected from the group consisting of melengesterol acetate, norgestomet, derivatives thereof, and mixtures thereof.

19. The implant of claim 1, wherein said pharmaceutical agent is an inorganic or organic macromolecular bioactive agent.

20. The implant of claim 19, wherein said bioactive agent is selected from the group consisting of acetylcholine esterase inhibitors, aminoglycocides, angiotensin-converting enzyme inhibitors, antiarrhythmics, antibacterial agents, anticancer agents, antidepressants, antidiabetics, antiepileptics, anti-viral agents, antihistamines, antihypertensives, antinauseants, antiprostaglandins, antirheumatics, antiseptics, barbiturates, beta-blockers, betalactamase inhibitors, calcium channel blockers, cardiac glycosides, cephalosporins, immune reagents, immunostimulators, immuno-suppressives, liposaccharide complexing agents, methylxanthines, minerals, O-beta-hydroxyethylated rutins, propxyphenes, salicyclates, tetracyclin compounds, vasodilators, acetaminiophen, acetazolamide, acetophenetidin, achromycine hydrochloride, bendofluazide, benzthiozide, betamethasone, calcium and salts, thereof including, leucovorin calcium, carbamazepine, clindamycin, chlorpropamide, chlorothalidone, chlorothiazide, clofibrate, cortisone acetate, cyclopenthiazide, dexamethazone, dextroamphetamine sulphate, diclofenac sodium, dioxin, dimethindene and salts thereof, diprophylline, disopyramide and salts thereof, dipyrone, doxycycline, fenbufen, fenoprofen, ferrous fumarate, flurbiprofen, frusemide, furosemide, glibenclamide, haloperidol, hydralazine, hydrochloride hydrochlorothiazide, hydroflumethiazide ibuprofen, indomethacin, indoprofen, iron salts, kanamycin, ketoprofen, L-Dopa, lithium salts, metaclopramide, methazolamide, methotrexate, fluoro-uracil, methotrexate sodium, methyl Dopa, metronidazole, minocyclin hydrochloride, mofebutazone, morphine, naproxen, nifedipine, oxyphenbutazone, penicillin, peridinol and salts thereof, phenylbutazone, phenobarbital, phenylpropanolamine, phenytoin, pindolol, piroxicam, pirprofen, potassium chloride, prazosin, propanolol, proxyphilline, pyrvinium emboate, quinidine, reserpine, salicylamide, salicyl salicyclic acid, sodium fluoride, spironolactone, sulfadiazine, sulfamerazine, tetracyclin compounds, tolbutamide, trihexylphenidyl hydrochloride, triethoprim, valproic acid, vancomycin, zoxazolamine, carbonic anhydrase inhibitors, anti-glaucoma agents, benzalkonium chloride, benzocaine, amilorid, hypnotics, sedatives, psychic energizers, tranquillizers, anticonvulsants, muscle relaxants, anti-Parkinson agents, analgesics, steroidal anti-inflammatories, anti-autoimmune agents, local and systemic anesthetics, contraceptives, sympathomimetrics, parasympathomimetrics, lipid regulating agents, anti-androgenics, antiparasitics, neoplastics, anti-AIDS agents, mutagens, teratogens, hypoglycaemic, nutritionals, fats, ophthalmics, otolaryngolmics, electrolytes, diagnostic agents, diuretics, nonsteroidal anti-inflammatories, antihistamines, chlomethazine, clemastine, hydroxyzine, terfenadine promethazine, astemizole, loratadine, mast cell stabilizers, bronchodilators, isoetharine hydrochloride, theophylline, albuterol, epinedrine, norepinedrine, adrenaline, noradrenaline, corticosteroids, prednisolone, hydrocortisone, cortisone acetate, flunisolide and triamincinolone acetate, anti cholesterol agents, estradiol, progesterones, testosterone, amino acids, thyroxine, peptides, histamines, fatty acids and fatty acid derivatives, inositol phosphates, gamma-aminobutyric acid, ketone bodies, acetylcholine, protein, DNA, carbohydrates, immunoglobulins G, M, A, D and E and their fragments and sub-chains, hormones, somatotropins, growth hormones, somatomedins, erythromycin, adrenocorticotropic hormone (ACTH), parahormone, follicle stimulating hormone, inhibin, renin, leuteinizing hormone, thyroid stimulating hormone, hypothalamic releasing hormones, TSH releasing factor, gastrointestinal hormones, vasopressin (ADH), somatostatin, immunomodulators, immunostimulators, immunoinhibitors colony stimulating factors (CSF), growth factors, cell chemotactic factors, antihemophilic factors, surface receptors and co-receptors, mineral oils, detergents, surfactants, derivatives thereof, and mixtures thereof.

21. The implant of claim 1 further comprising from about 0.1-50% by weight of an excipient.

22. The implant of claim 21, wherein said excipient is selected from the group consisting of starch, talc, glucose, sucrose, gelatin, malt, rice, flour, chalk, silica gel, calcium stearate, sodium stearate fumarate, stearic acid, sodium lauryl sulfonate, sodium stearate, glycerol monostearate, sodium chloride, polyethylene glycol, polyoxyethylene, glycerol behenate, hydrogenated vegetable oils, magnesium stearate, precipitated or fumed silicas, soidum starch glycolates, calcium phosphate, caldium carbonate, dextrins, polyvinyl pyrrolidone, polylactic acid, magnesium aluminum silicates, cellulose and its derivatives, especially ethylcellulose and microcrystalline cellulose, sodium carboxymethylcellulose, lactose, dried skim milk, derivatives thereof, and mixtures thereof.

23. The implant of claim 1 further comprising from about 0.1-50% by weight of an additive selected from the group consisting of inert and functional fillers, glidants, disintegrants, lubricants, adjuvants, antibiotic preservatives, polymeric supports, binders, coloring agents, and mixtures thereof.

24. The implant of claim 1 wherein said implant is a single pellet.

25. The implant of claim 1 wherein said implant comprises at least one discrete biological agent pellet and at least one discrete pharmaceutical agent pellet.

26. The implant of claim 24 wherein said pellet is selected from the group consisting of immediate release formulations, sustained release formulations, and mixtures thereof.

27. The implant of claim 25 wherein said discrete pellets are independently selected from the group consisting of immediate release formulations, sustained release formulations, and mixtures thereof.

28. The implant of claim 1 wherein said implant is suitable for subcutaneous implantation, vaginal administration, nasal administration, or sublingual administration.

29. The implant of claim 1 wherein said implant is suitable for subcutaneous implantation in an animal's ear.

30. A method for promoting growth in and/or immunizing animals comprising the steps of:
providing an implant including an effective amount of at least one biological agent, and an effective amount of at least one pharmaceutical agent;
administering said implant to an animal.

31. The method of claim 30, said effective amount of said biological agent being from about 1-100% by weight of said implant.

32. The method of claim 30, said effective amount of said biological agent being from about 50-99% by weight of said implant.

33. The method of claim 30, said effective amount of said biological agent being from about 60-75% by weight of said implant.

34. The method of claim 30, wherein said biological agent is a compound that stimulates an immune response in an animal.

35. The method of claim 34, wherein said biological agent is selected from the group consisting of vaccines, bacterins, toxoids, bacterin-toxoids, and mixtures thereof.

36. The method of claim 35, wherein said biological agent is selected from the group consisting of infectious bovine rhinotracheitis virus, bovine viral diarrhea virus, bovine parainfluenza 3 virus, bovine respiratory syncytial virus, Haemophilus somnus, Mannheimia haemolytica, Pasteurella multocida, Leptospira spp., Campylobacterfetus, Clostridium spp., rotavirus, coronavirus, Escherichia coli, Moraxella bovis, Bordetella bronchiseptica, Erysipelothrix rhusiopathiae, Actinobacillus pleuropneumoniae, Mycoplasma hyopneumoniae, Mycoplasma bovis, Mycoplasma dispar, porcine respiratory reproductive syndrome virus, porcine parvovirus, transmissible gastroenteritis virus, pseudorabies virus, Salmonella spp., Lawsonia ssp., Coccidia spp., Anaplasma spp., Babbesia spp., canine parvovirus, canine adenovirus, canine distemper, canine parainfluenza, rabies, feline leukemia, feline vital rhinotracheitis, feline calivirus, feline panleukopenia, Chlamydia psittaci, combinations and mixtures thereof.

37. The method of claim 30, said effective amount of said pharmaceutical agent being from about 1-100% by weight of said implant.

38. The method of claim 30, said effective amount of said pharmaceutical agent being from about 50-99% by weight of said implant.

39. The method of claim 30, said effective amount of said pharmaceutical agent being from about 60-75% by weight of said implant.

40. The method of claim 30, wherein said pharmaceutical agent is selected from the group consisting of parasiticides, pesticides, germicides, biocides, fungicides, insecticides, antioxidants, growth promotants, growth inhibitors, disinfectants, sterilization agents, catalysts, nutrients, vitamins, steroid hormones, prostaglandins, antibiotics, anti-inflammatory agents, chemotherapeutic agents, cardiovascular agents, antihypertensive agents, estrus suppressors, fertility promotors, somatotropins, gonadotropins, and mixtures thereof.

41. The method of claim 40, wherein said parasiticide is selected from the group consisting of polyketide ivermectins, milbemycins, milbemycin oximes, fenbendazole, oxfendazole, luferon, and mixtures thereof.

42. The method of claim 40, wherein said growth promotant is selected from the group consisting of progesterone, estradiol, estradiol benzoate, trenbolone acetate, zeranol, derivatives thereof, and mixtures thereof.

43. The method of claim 40, wherein said antibiotic is selected from the group consisting of macrolide antibiotics, penicillin, tetracycline, derivatives thereof, and mixtures thereof.

44. The method of claim 43, wherein said antibiotic is selected from the group consisting of tylosin, tylosin tartrate, oxytetracycline, neomycin, tilmicosin phosphate, ceftiofur hydrochloride, ceftiofur sodium, sulfadimethoxine, derivatives thereof, and mixtures thereof.

45. The method of claim 40, wherein said antibiotic is selected from the group consisting of bacteriostats, anti-inflammatory agents, and mixtures thereof.

46. The method of claim 40, wherein said steroid hormone is selected from the group consisting of levonorgestrel, estradiol 17β, testosterone, testosterone propionate, ethynyl estradiol, derivatives thereof, and mixtures thereof.

47. The method of claim 40, wherein said estrus suppressor is selected from the group consisting of melengesterol acetate, norgestomet, derivatives thereof, and mixtures thereof.

48. The method of claim 30, wherein said pharmaceutical agent is an inorganic or organic macromolecular bioactive agent.

49. The method of claim 48, wherein said bioactive agent is selected from the group consisting of acetylcholine esterase inhibitors, aminoglycocides, angiotensin-converting enzyme inhibitors, antiarrhythmics, antibacterial agents, anticancer agents, antidepressants, antidiabetics, antiepileptics, anti-viral agents, antihistamines, antihypertensives, antinauseants, antiprostaglandins, antirheumatics, antiseptics, barbiturates, beta-blockers, betalactamase inhibitors, calcium channel blockers, cardiac glycosides, cephalosporins, immune reagents, immunostimulators, immuno-suppressives, liposaccharide complexing agents, methylxanthines, minerals, O-beta-hydroxyethylated rutins, propxyphenes, salicyclates, tetracyclin compounds, vasodilators, acetaminiophen, acetazolamide, acetophenetidin, achromycine hydrochloride, bendofluazide, benzthiozide, betamethasone, calcium and salts, thereof including, leucovorin calcium, carbamazepine, clindamycin, chlorpropamide, chlorothalidone, chlorothiazide, clofibrate, cortisone acetate, cyclopenthiazide, dexamethazone, dextroamphetamine sulphate, diclofenac sodium, dioxin, dimethindene and salts thereof, diprophylline, disopyramide and salts thereof, dipyrone, doxycycline, fenbufen, fenoprofen, ferrous fumarate, flurbiprofen, frusemide, furosemide, glibenclamide, haloperidol, hydralazine, hydrochloride hydrochlorothiazide, hydroflumethiazide ibuprofen, indomethacin, indoprofen, iron salts, kanamycin, ketoprofen, L-Dopa, lithium salts, metaclopramide, methazolamide, methotrexate, fluoro-uracil, methotrexate sodium, methyl Dopa, metronidazole, minocyclin hydrochloride, mofebutazone, morphine, naproxen, nifedipine, oxyphenbutazone, penicillin, peridinol and salts thereof, phenylbutazone, phenobarbital, phenylpropanolamine, phenytoin, pindolol, piroxicam, pirprofen, potassium chloride, prazosin, propanolol, proxyphilline, pyrvinium emboate, quinidine, reserpine, salicylamide, salicyl salicyclic acid, sodium fluoride, spironolactone, sulfadiazine, sulfamerazine, tetracyclin compounds, tolbutamide, trihexylphenidyl hydrochloride, triethoprim, valproic acid, vancomycin, zoxazolamine, carbonic anhydrase inhibitors, anti-glaucoma agents, benzalkonium chloride, benzocaine, amilorid, hypnotics, sedatives, psychic energizers, tranquillizers, anticonvulsants, muscle relaxants, anti-Parkinson agents, analgesics, steroidal anti-inflammatories, anti-autoimmune agents, local and systemic anesthetics, contraceptives, sympathomimetrics, parasympathomimetrics, lipid regulating agents, anti-androgenics, antiparasitics, neoplastics, anti-AIDS agents, mutagens, teratogens, hypoglycaemic, nutritionals, fats, ophthalmics, otolaryngolmics, electrolytes, diagnostic agents, diuretics, nonsteroidal anti-inflammatories, antihistamines, chlomethazine, clemastine, hydroxyzine, terfenadine promethazine, astemizole, loratadine, mast cell stabilizers, bronchodilators, isoetharine hydrochloride, theophylline, albuterol, epinedrine, norepinedrine, adrenaline, noradrenaline, corticosteroids, prednisolone, hydrocortisone, cortisone acetate, flunisolide and triamincinolone acetate, anti cholesterol agents, estradiol, progesterones, testosterone, amino acids, thyroxine, peptides, histamines, fatty acids and fatty acid derivatives, inositol phosphates, gamma-aminobutyric acid, ketone bodies, acetylcholine, protein, DNA, carbohydrates, immunoglobulins G, M, A, D and E and their fragments and sub-chains, hormones, somatotropins, growth hormones, somatomedins, erythromycin, adrenocorticotropic hormone (ACTH), parahormone, follicle stimulating hormone, inhibin, renin, leuteinizing hormone, thyroid stimulating hormone, hypothalamic releasing hormones, TSH releasing factor, gastrointestinal hormones, vasopressin (ADH), somatostatin, immunomodulators, immunostimulators, immunoinhibitors colony stimulating factors (CSF), growth factors, cell chemotactic factors, antihemophilic factors, surface receptors and co-receptors, mineral oils, detergents, surfactants, derivatives thereof, and mixtures thereof.

50. The method of claim 30 further comprising from about 0.1-50% by weight of an excipient.

51. The method of claim 50, wherein said excipient is selected from the group consisting of starch, talc, glucose, sucrose, gelatin, malt, rice, flour, chalk, silica gel, calcium stearate, sodium stearate fumarate, stearic acid, sodium lauryl sulfonate, sodium stearate, glycerol monostearate, sodium chloride, polyethylene glycol, polyoxyethylene, glycerol behenate, hydrogenated vegetable oils, magnesium stearate, precipitated or fumed silicas, soidum starch glycolates, calcium phosphate, caldium carbonate, dextrins, polyvinyl pyrrolidone, polylactic acid, magnesium aluminum silicates, cellulose and its derivatives, especially ethylcellulose and microcrystalline cellulose, sodium carboxymethylcellulose, lactose, dried skim milk, derivatives thereof, and mixtures thereof.

52. The method of claim 30 further comprising from about 0.1-50% by weight of an additive selected from the group consisting of inert and functional fillers, glidants, disintegrants, lubricants, adjuvants, antibiotic preservatives, polymeric supports, binders, coloring agents, and mixtures thereof.

53. The method of claim 30 wherein said implant is a single pellet.

54. The method of claim 30 wherein said implant comprises at least one discrete biological agent pellet and at least one discrete pharmaceutical agent pellet.

55. The method of claim 53 wherein said pellet is selected from the group consisting of immediate release formulations, sustained release formulations, and mixtures thereof.

56. The method of claim 54 wherein said discrete pellets are independently selected from the group consisting of immediate release formulations, sustained release formulations, and mixtures thereof.

57. The method of claim 30 wherein said administering step is selected from the group consisting of subcutaneous implantation, vaginal administration, nasal administration, or sublingual administration.

58. The method of claim 30 wherein said administering step is subcutaneous implantation in an animal's ear.
